(19) United States
(12) Patent Application Publication
(10) Pub. No.: US 2011/0152857 A1
(43) Pub. Date: Jun. 23, 2011

(54) Apparatus and Methods For Electrophysiology Procedures

(76) Inventor: Frank Ingle, Palo Alto, CA (US)

(21) Appl. No.: 12/916,502

(22) Filed: Oct. 30, 2010

Related U.S. Application Data
(60) Provisional application No. 61/288,275, filed on Dec. 19, 2009.

(51) Int. Cl. A61B 18/12

(52) U.S. Cl. 606/40

(57) ABSTRACT

Methods and apparatus in accordance with at least some of the present disclosure employ a measured heat transfer property to evaluate electrode/tissue contact. Methods and apparatus in accordance with at least some of the present disclosure employ the relationship between impedance measurements and sub-surface temperature to control power.

### CROSS-REFERENCE TO RELATED APPLICATIONS

This application claims the benefit of U.S. Provisional Application Ser. No. 61/288,275, filed Dec. 19, 2009 and entitled “Apparatus and Methods for Electrophysiology Procedures,” which is incorporated herein by reference.

### BACKGROUND

1. Field

The present application relates generally to electrophysiology procedures including, for example, ablation procedures that form lesions in tissue.

2. Description of the Related Art

There are many instances where electrodes are inserted into the body. One instance involves the treatment of cardiac conditions such as atrial fibrillation, atrial flutter and ventricular tachycardia, which lead to an unpleasant, irregular heart beat, called arrhythmia. Atrial fibrillation, flutter and ventricular tachycardia occur when anatomical obstacles in the heart disrupt the normally uniform propagation of electrical impulses in the atria. These anatomical obstacles (called “conduction blocks”) can cause the electrical impulse to degenerate into several circular wavelets that circulate about the obstacles. These wavelets, called “reentry circuits,” disrupt the normally uniform activation of the chambers within the heart.

A variety of minimally invasive electrophysiological procedures employing catheters that carry one or more electrodes have been developed to treat conditions within the body by ablating soft tissue (i.e. tissue other than blood and bone). Soft tissue is simply referred to as “tissue” herein and references to “tissue” are not references to blood. With respect to the heart, minimally invasive electrophysiological procedures have been developed to treat atrial fibrillation, atrial flutter and ventricular tachycardia by forming therapeutic lesions in heart tissue. The formation of lesions by the coagulation of soft tissue (also referred to as “ablation”) during minimally invasive surgical procedures can provide the same therapeutic benefits provided by certain invasive, open-heart surgical procedures. In particular, the lesions may be placed so as to interrupt the conduction routes of reentry circuits.

The catheters employed in electrophysiological procedures typically include a relatively long and relatively flexible shaft that carries a distal tip electrode and, in some instances, one or more additional electrodes near the distal end of the catheter. The proximal end of the catheter shaft is connected to a handle which may or may not include steering controls for manipulating the distal portion of the catheter shaft. The length and flexibility of the catheter shaft allow the catheter to be inserted into a main vein or artery (typically the femoral artery), directed into the interior of the heart where the electrodes contact the tissue that is to be ablated. Fluoroscopic imaging may be used to provide the physician with a visual indication of the location of the catheter. Exemplary catheters are disclosed in U.S. Pat. Nos. 6,013,052, 6,203,525, 6,214,002 and 6,241,754.

The tissue coagulation energy is typically supplied and controlled by an electrosurgical unit (“ESU”) during the therapeutic procedure. More specifically, after an electrophysiology device has been connected to the ESU, and one or more electrodes or other energy transmission elements on the device have been positioned adjacent to the target tissue, energy from the ESU is transmitted through the electrodes to the tissue to from a lesion. The amount of power required to coagulate tissue ranges from 5 to 150 W. The energy may be returned by an electrode carried by the therapeutic device, or by an indifferent electrode such as a patch electrode that is secured to the patient's skin.

The present inventor has determined that electrode/tissue contact is an important issue, for reasons of efficiency and safety. Poor electrode/tissue contact with the target tissue, and/or the absence of electrode/tissue contact, increases the amount of ablation energy that is transmitted into the surrounding tissue and blood. With respect to efficiency, the corresponding reduction in the amount of energy that is transmitted to the target tissue reduces the likelihood that a transmural, or otherwise therapeutic, lesion will be formed. Poor electrode/tissue contact can also increase the amount of time that it takes to complete the procedure. Turning to safety, transmission of excessive amounts of energy into the surrounding tissue can result in the formation of lesions in non-target tissue which, in the exemplary context of the treatment of cardiac conditions, can impair heart function. The transmission of excessive amounts of energy into the blood can result in the formation of coagulum and emboli. It also increases the amount of energy that is returned by the patch electrode, which can result in skin burns. Even when the level of electrode/tissue contact is at or above the minimum level required for safe and effective ablation, different types of lesions call for different levels of electrode/tissue contact. Accordingly, the present inventor has determined that it would be desirable to provide reliable methods and apparatus for determining whether or not an electrode is in contact with tissue and, if so, the level of contact, prior to the application of ablation energy.

It is also important to keep the sub-surface tissue temperature below 100° C. during ablation procedures. Sub-surface tissue temperatures at or above 100° C. will cause liquid within the sub-surface tissue to vaporize and expand. Ultimately, the tissue will tear or pop, which will result in perforations of the epicardial or other tissue surface and/or the dislodging of chunks of tissue that can cause strokes. Many conventional electrophysiology systems rely on temperature measurements taken by a sensor (e.g. a thermocouple or thermistor) on an electrode that is delivering ablation energy. The present inventor has determined that there are a number of issues associated with the temperature measurements from temperature sensors that are carried on electrodes, as well as the power control methodologies based thereon. For example, electrode based temperature sensors do not measure sub-surface temperature, which may be higher than surface temperatures. The temperature of the electrode may also be subject to convective cooling due to blood flow, especially when a long electrode tip is employed, and the amount of cooling depends on the local blood velocity. Accordingly, the present inventor has determined that it would be desirable to provide reliable methods and apparatus for measuring sub-surface tissue temperatures that do not rely on electrode based temperature sensors.

### SUMMARY

Methods and apparatus in accordance with at least some of the present inventions employ a measured heat transfer property to evaluate electrode/tissue contact. Such methods and apparatus provide a number of advantages over conventional methods and apparatus. For example, the present methods and apparatus allow the clinician to determine whether or not there is an adequate level of electrode/tissue contact prior to deciding whether or not to initiate the transmission of energy to tissue.

Methods and apparatus in accordance with at least some of the present inventions employ the relationship between impedance measurements and sub-surface temperature to control power. Such methods and apparatus provide a number of advantages over conventional methods and apparatus. For example, the impedance measurements more accurately represent the sub-surface tissue temperature than temperature measurements taken by sensors on the electrode delivering the ablation energy and, therefore, allow sub-surface tissue temperature to be more accurately controlled.

The above described and many other features and attendant advantages of the present inventions will become apparent as the inventions become better understood by reference to the following detailed description when considered in conjunction with the accompanying drawings.

### BRIEF DESCRIPTION OF THE DRAWINGS

Detailed description of exemplary embodiments will be made with reference to the accompanying drawings.

FIG. 1 is a perspective view of an electrophysiology system in accordance with one embodiment of a present invention.

FIG. 2 is a section view taken along line 2-2 in FIG. 1.

FIG. 3 is a section view taken along line 3-3 in FIG. 1.

FIG. 4 is a partial section view of a portion of the exemplary electrophysiology system illustrated in FIG. 1.

FIGS. 5-7 are side, partial section views illustrating various level of electrode/tissue contact.

FIG. 8 is a flow chart in accordance with at least one embodiment of a present invention.

FIG. 9 is a side, partial section view of tissue and an electrode in accordance with one embodiment of a present invention.

FIG. 10 is a plan view of a catheter apparatus in accordance with one embodiment of a present invention.

FIG. 11 is a side view of a portion of the exemplary catheter apparatus illustrated in FIG. 10.

FIG. 12 is a side, partial section view of a portion of the exemplary catheter apparatus illustrated in FIG. 10.

FIG. 13 is a side view of an electrode in accordance with one embodiment of a present invention.

FIG. 14 is a flow chart in accordance with at least one embodiment of a present invention.

FIG. 15 is a flow chart in accordance with at least one embodiment of a present invention.

### DETAILED DESCRIPTION OF EXEMPLARY EMBODIMENTS

The following is a detailed description of the best presently known modes of carrying out the inventions. This description is not to be taken in a limiting sense, but is made merely for the purpose of illustrating the general principles of the inventions.

The present inventions have application in the treatment of conditions within the heart, gastrointestinal tract, prostrate, brain, gall bladder, uterus, and other regions of the body. With regard to the treatment of conditions within the heart, the present inventions may be associated with the creation of lesions to treat atrial fibrillation, atrial flutter and ventricular tachycardia.

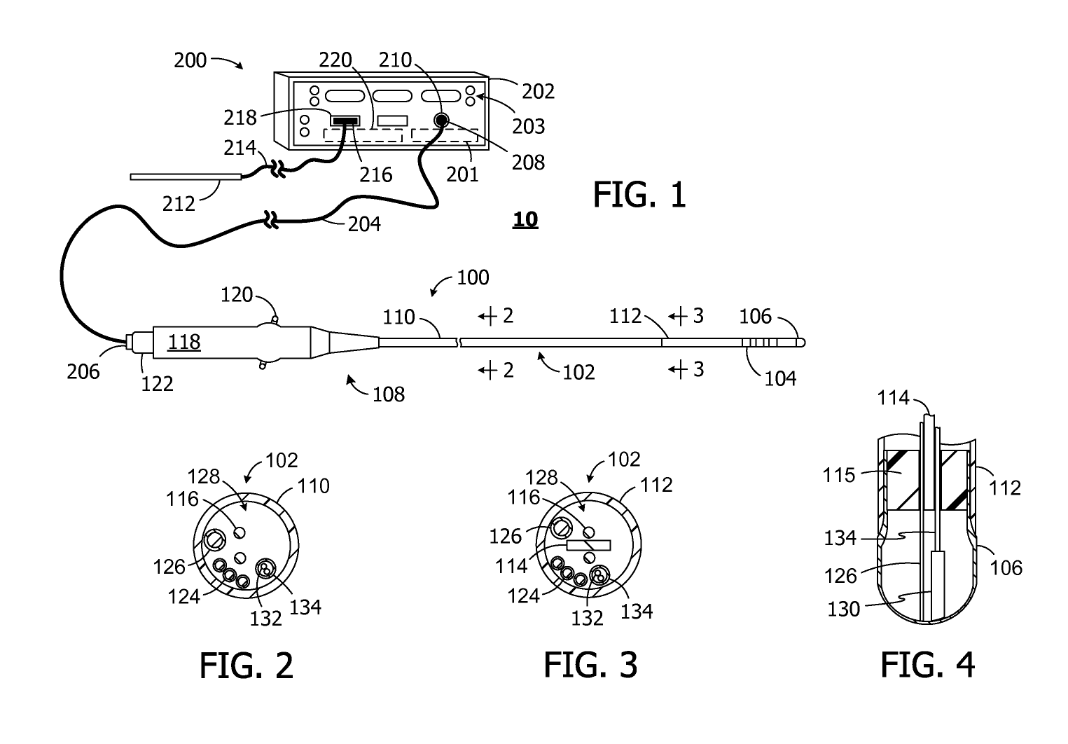

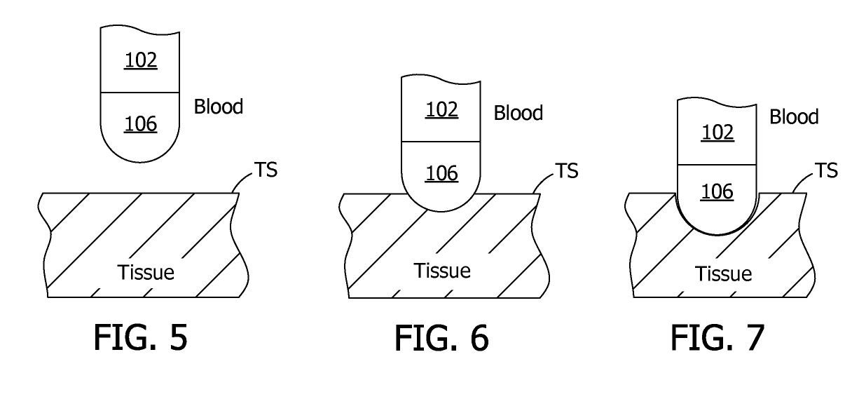

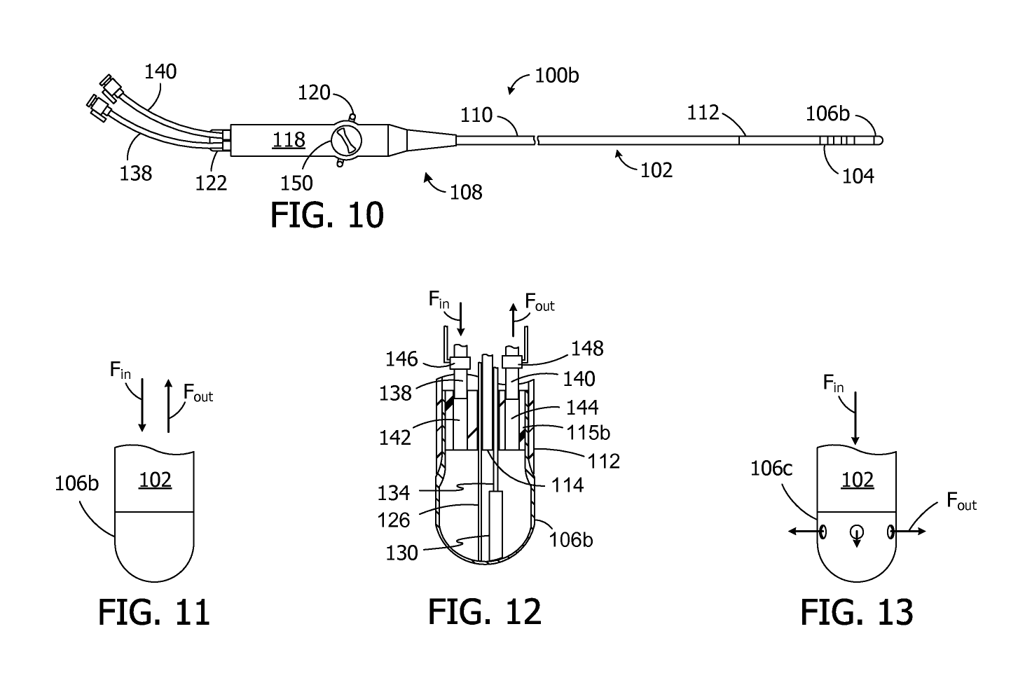

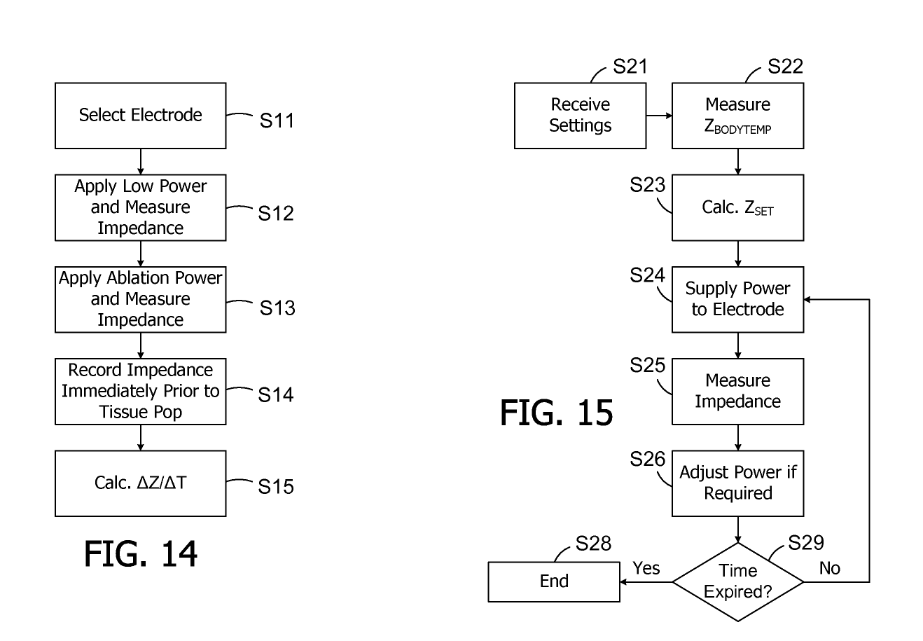

An exemplary electrophysiology system 10 which may embody or otherwise be associated with at least some of the present inventions is illustrated in FIGS. 1-4. The exemplary system 10 includes a catheter apparatus 100 and a power supply and control apparatus 200. The tissue coagulation system 10 may be used to perform tissue ablation procedures to create lesions in tissue surfaces. As discussed in greater detail below with reference to FIGS. 5-8, the exemplary power supply and control apparatus 200 is configured to determine whether or not the catheter apparatus ablation electrode is in contact with tissue, and to determine the level of electrode/tissue contact, based on a heat transfer property measured at or near the target tissue. The exemplary power supply and control apparatus 200 is also configured to determine the sub-surface tissue temperature based on a change in tissue impedance, as is discussed below with reference to FIGS. 14 and 15, and to control power to the ablation electrode based on the determined sub-surface temperature. It should be noted that the system illustrated in FIGS. 1-4 is merely one example of an electrophysiology system with which the present inventions may be associated. The present inventions are applicable to, for example any and all tissue coagulations systems, including those yet to be developed and those that are not catheter based, as well as to the individual components thereof. For example, the present inventions are also applicable to tissue ablation systems that employ fluid to cool the ablation electrode, as is discussed below with reference to FIGS. 10-13.

The exemplary catheter apparatus 100 illustrated in FIGS. 1-4 includes a hollow, flexible catheter 102, a plurality of ring electrodes 104, a tip electrode 106, and a handle 108. The catheter 102 may be steerable and formed from two tubular parts, or members, both of which are electrically non-conductive. The proximal member 110 is relatively long and is attached to the handle 108, while the distal member 112, which is relatively short, carries the electrodes 104 and 106. The exemplary catheter 102 is also configured for use within the heart and, accordingly, is about 6 French to about 10 French in diameter and the portion that is inserted into the patient is typically about 60 to 160 cm in length. The exemplary catheter apparatus 100 is steerable and, to that end, is provided with a conventional steering center support and steering wire arrangement. The proximal end of the exemplary steering center support 114 is mounted near the distal end of the proximal member 110, while the distal end of the steering center support is secured to the tip electrode 106 with an anchor 115. A pair of steering wires 116 are secured to opposite sides of the steering center support 114 and extend through the catheter body 102 to the handle 108, which is also configured for steering. More specifically, the exemplary handle 108 includes a handle body 118 and a lever 120 that is rotatable relative to the handle body. The proximal end of the catheter 102 is secured to the handle body 118, while the proximal ends of the steering wires 116 are secured to the lever 120. Rotation of the lever 120 will cause the catheter distal member 112 to deflect relative to the proximal member 110.

The exemplary ring electrodes 104, which may be used for electrical sensing or tissue ablation, are connected to an electrical connector 122 on the handle 108 by signal wires 124. Electrically conducting materials, such as silver, platinum, gold, stainless steel, plated brass, platinum iridium and combinations thereof, may be used to form the electrodes 104. The diameter of the exemplary electrodes 104 will typically range from about 5 French to about 11 French, while the length is typically about 1 mm to about 4 mm with a spacing of about 1 mm to about 10 mm between adjacent electrodes. The exemplary tip electrode 106 may be formed from any suitable electrically conductive material. By way of example, but not limitation, suitable materials for the tip electrode 106 include silver, platinum, gold, stainless steel, plated brass, platinum iridium and combinations thereof. The tip electrode 106 may be generally cylindrical in shape with a hemispherical end and, in some exemplary implementations sized for use within the heart, may be from about 5 French to about 11 French in diameter and about 3 mm to about 8 mm in length. Power for the tip electrode 106 is provided by a power wire 126 that is soldered to a portion of the tip electrode and extends through the catheter lumen 128 to the electrical connector 122 on the handle 108.

With respect to the temperature sensing performed by the exemplary catheter apparatus 100, a temperature sensor 130 is mounted in the tip electrode 106. In the illustrated embodiment, the temperature sensor 130 is a thermocouple. The thermocouple wires 132 from the thermocouple extend through tube 134 to the electrical connector 122. Other types temperatures sensors, such as thermistors, may also be employed.

The exemplary power supply and control apparatus (“power supply”) 200 includes an electrosurgical unit (“ESU”) 202 that supplies and controls RF power. A suitable ESU is the Model 4810A ESU sold by Boston Scientific Corporation of Natick, Mass. The ESU 202 has a power generator 201 and a control panel 203 that allows the user to, for example, set the power level, the duration of power transmission, and a tissue temperature for a given coagulation procedure. The ESU 202 may also be configured to measure a heat transfer property at the tip electrode 106 and determine the level of electrode/tissue contact based on the heat transfer property. The ESU 202 may also be configured to measure impedance, correlate changes in measured impedance to changes in sub-surface tissue temperature, and control power to the electrode 106 based on the changes in impedance.

The ESU 202 transmits energy to the electrode 106 by way of a cable 204. The cable 204 includes a connector 206 which may be connected to the catheter electrical connector 122 which, in turn, is connected to the catheter apparatus power and signal wires 124, 126 and 132. The cable 204 also includes a connector 208, which may be connected to a power output port 210 on the ESU 202. Power to the catheter apparatus 100 may be maintained at a constant level during a coagulation procedure, or may be varied, or may substantially reduced or may be shut off completely, depending upon the temperatures measured at the tip electrode 106 with the temperature sensor 130 and/or measured impedance. The exemplary ESU 202 is capable of performing both unipolar and bipolar tissue coagulation procedures. During unipolar procedures performed with the exemplary system 10 illustrated in FIG. 1, tissue coagulation energy emitted by the electrode 106 is returned to the ESU 202 through an indifferent electrode 212 that is externally attached to the skin of the patient with a patch and a cable 214. The cable 214 includes a connector 216 that may be connected to one of the power return ports 218 on the ESU 202. Preferably, the ESU power output port 210 and corresponding connector 208 have different configurations than the power return port 218 and corresponding connectors 216 in order to prevent improper connections.

The exemplary ESU 202 also includes a controller 220, such as a microprocessor, microcontroller or other control circuitry, that controls the power delivered to the catheter apparatus in accordance with parameters and instructions stored in a programmable memory unit (not shown). Suitable programmable memory units include, but not limited to, FLASH memory, random access memory (“RAM”), dynamic RAM (“DRAM”), or a combination thereof. A data storage unit, such as a hard drive, flash drive, or other non-volatile storage unit, may also be provided. The controller 220 can employ proportional control principles, adaptive control, neural network, or fuzzy logic control principles. In the illustrated implementation, proportional integral derivative (PID) control principles are applied. The controller 220 may be used to perform, for example, conventional temperature and power control functions, as well as the methods and functions described below with reference to FIGS. 5-15 and set forth in the claims.

Turning to the electrode/tissue contact sensing aspects of at least some of the present inventions, FIGS. 5-7 illustrate the contact states discussed below. FIG. 5 shows the catheter 102 positioned such that electrode of interest, i.e. the tip electrode 106 of the exemplary catheter apparatus 100, in spaced relation to the tissue surface TS and entirely within the blood pool. FIG. 6 shows the catheter 102 positioned such that the tip electrode 106 is lightly touching the tissue, while FIG. 7 shows the catheter 102 positioned such that the tip electrode 106 is firmly pressed into the tissue. In the exemplary context of the left atrium, the amount of force associated with light touching of the tissue may be about 0.015 kilograms (0.147 newtons), while the amount of force associated firm pressing into the tissue may be about 0.030 kilograms (0.294 newtons).

The present inventor has determined that the difference between the heat transfer properties of blood and the heat transfer properties of tissue may be used to determine whether an electrode is in contact with blood or tissue and, when in contact with tissue, whether the electrode is lightly touching tissue or is firmly pressed against tissue. The determination may be made before the ablation energy is supplied, in order to confirm that an appropriate level of contact has been achieved, as well as after the application of ablation energy is initiated, in order to confirm that the appropriate level of contact has been maintained.

One heat transfer property that may be used to make contact determinations is thermal resistance at the electrode, which is related to the geometry of the electrode and the tissue thermal resistivity. Thermal resistance is a measure of a physiological body's ability to prevent heat from flowing through it, and is equal to the change in temperature of the electrode divided by the power supplied to the electrode. The formula is R=ΔT/P, and the SI unit of measurement is ° C./W. The thermal resistance of tissue is relatively high as compared to flowing or stationary blood, although the difference is greater when the blood is flowing due to the addition of convective cooling. In other words, blood is a better thermal conductor than tissue and flowing blood is a better thermal conductor than stationary blood. Accordingly, when an electrode supplies power only to blood (FIG. 5), the temperature increase measured at the electrode will be less than the temperature increase associated with the electrode supplying the same amount of power to tissue (FIGS. 6 and 7). The greater the percentage of the electrode surface that is in contact with blood, as compared to the percentage of the electrode surface that is in contact with tissue, the lower the measured temperature increase of the electrode will be when power is supplied. Accordingly, the temperature increase measured at a power supplying electrode that is lightly touching tissue and has a relatively high percentage of the it's surface is in contact with blood (FIG. 6) will be less than the temperature increase of an electrode supplying the same amount of power that is firmly pressed into tissue (FIG. 7) and has a relatively low percentage of the it's surface is in contact with blood.

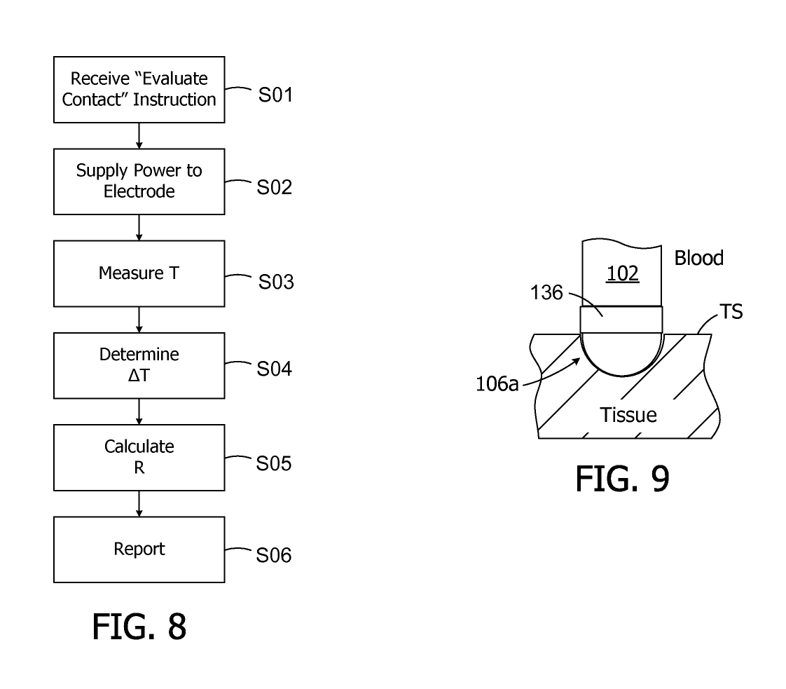

Turning to FIG. 8, the exemplary controller 220 may be configured to operate the ESU 202 as follows. After the clinician has advanced the catheter 102 to the point at which one would expect the tip electrode 106 to be in contact with the target tissue, an “evaluate contact” instruction from the clinician may be received by way of the control panel 203 (Step S01). The controller 220 will begin the evaluation process by controlling the ESU 202 to supply a predetermined amount of power P to the tip electrode 106 for a predetermined period (Step S02). The power level and time period should both be relatively low, i.e. too low to ablate tissue during the evaluation period. In one exemplary implementation, about 1 W is supplied for 2-5 seconds. The temperature sensor 130 may then be used to measure the temperature of the tip electrode 106 (Step S03) during the application relatively low power. The measured temperature Tm is then used to calculate the electrode temperature increase ΔT_{ELECTRODE} associated with the supplied power P (Step S04). The temperature prior to supplying power is assumed to be body temperature (37° C.) in the exemplary implementation and, accordingly, the increase in temperature ΔT_{ELECTRODE} equals the measured temperature T_{M} minus 37, i.e. ΔT_{ELECTRODE}=T_{M}−37. In other implementations, the temperature sensor 130 may be used to take measure electrode temperature prior to the application of power. Thermal resistance is then calculated using the R=ΔT_{ELECTRODE}/P formula (Step S05), and the results of the thermal resistance calculations are reported (Step S06).

The thermal resistance may be reported in a variety of ways. For example, a display on the ESU control panel 203 may be used to display the value of the thermal resistance. Here, the clinician could simply rely on his or her own experience, and/or other information, to determine whether or not the calculated thermal resistance indicates that the electrode 106 is in completely within the blood pool, is lightly touching the tissue, or is firmly pressed into the tissue. Alternatively, or in addition, the ESU controller 220 may be provided with a lookup table that stores thermal resistance values (or ranges of values) for particular electrode configurations and, in some instances, stores thermal resistance values (or ranges of values) for particular electrode configurations on a tissue region by tissue region basis. For example, one set of stored values could be associated with a 7 French tip electrode with a hemispherical end generally, or could be associated with a 7 French tip electrode with a hemispherical end being used in the left atrium in particular. Such values may be experimentally derived or approximated by calculations. The electrode configuration being employed may be input by way of the ESU control panel 203 or may be automatically determined when a catheter apparatus with an identification instrumentality is plugged into the ESU. In either case, the ESU controller 220 will compare the measured thermal resistance to the stored values for the particular electrode and, in some instances, the particular electrode and particular tissue region, and determine whether the measured thermal resistance corresponds to the electrode being completely in the blood pool, lightly touching tissue, or firmly pressed into tissue. The results of this analysis may be audibly or visibly reported to the clinician by way of the control panel 203.

It should be noted here that the fidelity of thermal resistance based determinations of electrode/tissue contact may the improved by thermally insulating the portions of the electrode that are not expected to be in contact with tissue when the electrode is properly oriented and firmly pressed into tissue. The tip electrode 106a illustrated in FIG. 9, for example, includes a layer of thermal insulation 136 on the cylindrical portion of the electrode. The insulation prevents (or at least substantially reduces) heat transfer from the electrode 106a to the blood when the electrode is firmly pressed into tissue and reduces the amount of heat transfer from the electrode 106a to the blood when the electrode is lightly pressed into tissue, thereby amplifying the difference between the measured thermal resistance with the electrode 106a is entirely within the blood pool and the measured thermal resistance with the electrode pressed into tissue.

Thermal resistance may also be used to evaluate tissue electrode/tissue contact in electrophysiology systems that employ fluid cooled tip electrodes, including closed tips where the fluid returns to the fluid source and open tips where the fluid flow through the tip. An exemplary catheter apparatus 100b with a closed tip electrode 106b is illustrated in FIGS. 10-12. The catheter apparatus 100b is substantially similar to the catheter apparatus 100 and similar elements are represented by similar reference numerals.

Here, however, cooling fluid inlet and outlet tubes 138 and 140 extend though the handle 108 to inlet and outlet lumens 142 and 144 in an anchor 115a. The inlet and outlet 138 and 140 tubes may be connected to a fluid source in conventional fashion. Examples of such fluid sources are disclosed in, for example, U.S. Pat. No. 6,939,350, which is incorporated herein by reference. Temperature sensing collars 146 and 148, which position temperature sensors (not shown) in the flow path of the incoming and outgoing cooling fluid, are also provided. The temperature sensors sense the incoming temperature of the cooling fluid T_{IN} and the outgoing temperature of the cooling fluid T_{OUT}. A fluid control knob 150, and a valve, may also be provided on the handle 118.

The power supplied to tissue from a cooled electrode, P_{TISSUE}, is equal to the power P supplied to the electrode less the portion of power that is lost to, and heats, the cooling fluid F, P_{LOST}. The power lost to the cooling fluid may be determined by measuring the temperature of the fluid as it enters the tip electrode and the temperature of the fluid as it exits the tip electrode. In particular, the power lost to the cooling fluid, P_{LOST}=ΔT_{FLUID}×Q×ρ×Cp, where ΔT_{FLUID} is T_{OUT}-T_{IN}, Q is the flow rate, ρ is the fluid density, and Cp is the fluid heat capacity. The fluid density and fluid heat capacity of various cooling fluids may be stored in the ESU controller 220, or may be input by way of the control panel 203, or may be supplied to the ESU directly from the fluid supply apparatus. The flow rate may be input into the ESU controller by way of the control panel 203 or may be supplied to the ESU directly from the fluid supply apparatus. Thermal resistance may be calculated by the ESU controller using the R=(ΔT_{ELECTRODE})/(P-P_{LOST}) formula. The calculated thermal resistance may be used to make an electrode/tissue contact determination in the manner discussed above.

An exemplary catheter apparatus with an open tip electrode 106c is illustrated in FIG. 13. Thermal resistance may be calculated in a manner similar to that described above with reference to the closed tip 106b. Here, however, T_{OUT} may be measured indirectly by, for example, measuring the temperature of a portion of the tip electrode 106c that is the same temperature as the fluid when the fluid exits the electrode.

It should also be noted here that thermal resistance is not the only measurable heat transfer property that may be used to make tissue contact determinations. The heat transfer coefficient may also be used. Thermal resistance and/or heat transfer coefficient may also be employed during an ablation procedure to evaluate electrode/tissue contact.

It should also be noted here that the use of measured thermal resistance to determine electrode/tissue contact is not limited to tip electrodes with a hemispherical end surface. The principles described above are also applicable to, for example tip electrodes with other shapes and electrodes that are located proximal of the tip, such as ring and coil electrodes.

Turning to FIGS. 14 and 15, at least some of the present inventions also include methods and apparatus for preventing sub-surface tissue temperatures from reaching levels that will result in tissue popping. For example, at least some of the present inventions include methods and apparatus for maintaining tissue temperature about 1-2 mm below the tissue surface at a preselected level that is suitable for ablation but below that which will result in tissue popping. Changes in measured tissue electrical impedance (which is referred to in this context simply as “impedance”) may be used to represent changes in sub-surface temperature. In particular, the measured reduction in tissue impedance during an ablation procedure may be compared to expected impedance reduction for a particular electrode configuration and target tissue region. The relationship between the measured impedance reduction and the expected impedance reduction may be use to, for example, control power in such a manner that the sub-surface tissue temperature remains at a preselected value suitable for ablation (e.g. 65° C.), and below the temperature at which popping will occur (about 100° C.), during an ablation procedure.

With respect to impedance itself, impedance is a complex quantity comprised of a real part called resistance and an imaginary part called reactance. Reactance is essentially zero at the typical operating frequencies of RF generators (e.g. 500 KHz) and, accordingly, impedance is essentially equal to resistance. As such, impedance and resistance may be considered to be equivalents in the context of RF ablation, and impedance may be measured by, for example, simply measuring current and voltage and dividing voltage by current.

The expected impedance reduction for a particular electrode configuration and target tissue region may be based on empirical data or theoretical calculations. Referring first to FIG. 14, empirical data may be generated as follows in vitro with tissue samples or in vivo using animal testing. A catheter with a particular electrode configuration may be selected (Step S11). The electrode may then be used to measure the impedance of a tissue sample at body temperature (37° C.) Z_{BODYTEMP} (Step S12). To that end, tissue impedance at body temperature Z_{BODYTEMP} may be measured by applying a power to the tissue by way of the electrode at a low level (e.g. about 1 W) for about 2-5 seconds. As used in the impedance measurement context herein, a “low” level of power is a level of power that will not cause a temperature increase of more than 10° C. so that tissue will not be ablated. This measurement establishes the pre-ablation body temperature impedance of the tissue. Next, power is applied to the tissue at level that will result in the sub-surface temperature increasing to the point at which the tissue pops (e.g. about 100° C.) within a time limit commonly associated with ablation procedure (e.g. 30 seconds) (Step S13). For example, 20-40 W may be applied until the tissue pops. The impedance, which will drop as power is applied and the sub-surface temperature increases, is measured during the application of power. The impedance level immediately prior to the pop Z_{POP}, and the corresponding increase in impedance from Z_{BODYTEMP}, is recorded (Step S14).

It should also be noted that empirical data may be obtained by recording the data described above during actual in vivo ablation procedures on humans and, in those instances where the ablation procedure results in a tissue pop, noting the values of Z_{BODYTEMP} and Z_{POP}.

The present inventor has determined that impedance decreases with the increase in sub-surface tissue temperature in generally linear fashion prior to being coagulated. The increase in sub-surface tissue temperature and corresponding reduction in tissue impedance prior to the tissue pop may be used to derive an impedance reduction to temperature increase ratio ΔZ/ΔT for particular tissue types and electrode configurations. Assuming that the sub-surface temperature at the time of tissue popping is 100° C., the ΔZ/ΔT ratio would be equal to (Z_{BODYTEMP}−Z_{POP})/(37° C.-100° C.). In one numerical example, Z_{BODYTEMP}=150 Ohms and Z_{POP}=120 Ohms and, accordingly, the AZ/AT ratio is equal to about −0.5 Ohms/° C. A sub-surface tissue temperature increase from body temperature to one exemplary ablation temperature, i.e. from 37° C. to 65° C., would result in an expected tissue impedance reduction Z_{DROP} of 14 Ohms given the linear aspect of the impedance decrease.

The ΔZ/ΔT ratio may be used to create a set point for the control of ablation procedures that is more representative of sub-surface tissue temperatures than temperature measurements taken at the tissue surface. More specifically, the ΔZ/ΔT ratio may be used to select an ablation procedure impedance reduction that corresponds to the desired sub-surface temperature increase. Using the numerical example presented in the preceding paragraph, where ΔZ/ΔT=−0.5 Ohms/° C., a sub-surface temperature set point T_{SET} of 65° C. would correspond to a 28° C. temperature increase, i.e. from 37° C. to 65° C., and an impedance reduction Z_{DROP} equal to 14 Ohms. The impedance set point Z_{SET} is equal to Z_{BODYTEMP}−Z_{DROP}. Again using the numerical example presented in the preceding paragraph, the impedance set point Z_{SET}=150 Ohms−14 Ohms=136 Ohms. The ΔZ/ΔT ratio for various for various tissue types and electrode configurations may be stored by ESU controller 220 or some other portion of the ESU.

The difference, if any, between the impedance measured during the ablation procedure Z_{PROCEDURE} may be compared to the impedance set point Z_{SET} during an ablation procedure and used by the ESU controller 220 regulate the power supplied to the electrode. For example, and as alluded to above, the ESU controller 220 may employ proportional integral derivative (PID) control principles, proportional control principles, adaptive control principles, neural network control principles, or fuzzy logic control principles to control power as a function of ablation procedure impedance Z_{PROCEDURE} and the impedance set point Z_{SET}. As a result of such regulation, the level of power to the electrode may be increased in some instances where the impedance measured during the ablation procedure Z_{PROCEDURE} is greater than the impedance set point Z_{SET}, the level of power to the electrode may be decreased in some instances where the impedance measured during the ablation procedure Z_{PROCEDURE} is less than the impedance set point Z_{SET}, and the level of power to the electrode may be maintained in some instances where the impedance measured during the ablation procedure Z_{PROCEDURE} is equal to (or substantially equal to) the impedance set point Z_{SET}. There are a variety of advantages associated with controlling power in this manner. For example, as compared to controlling power based on temperature measured at the power supplying electrode, controlling power as a function of ablation procedure impedance Z_{PROCEDURE} and the impedance set point Z_{SET} results in better control of the temperature of sub-surface tissue.

The impedance set point Z_{SET} may be provided in a variety of ways. By way of example, but not limitation, the ESU controller 220 may be configured to receive a sub-surface temperature set point T_{SET} by way of the control panel 203. Electrode configuration (e.g. size and shape) may also be input way of the control panel 203 or may be automatically determined by the ESU controller 220 when a catheter apparatus, such as the exemplary catheter apparatus 100, is plugged into the ESU 200. In those instances where the ΔZ/ΔT ratios are stored for various electrode configurations, the ESU controller 220 will calculate ΔT by either subtracting the assumed body temperature (37° C.) from the sub-surface temperature set point T_{SET}, or in those instances where body temperature is measured prior to the ablation procedure, by subtracting the measured body temperature from the sub-surface temperature set point T_{SET}. The ESU controller 220 may then apply the appropriate ΔZ/ΔT ratio to ΔT to calculate Z_{DROP} which, in turn, may be used to calculate the impedance set point Z_{SET} in the manner described above and below. In other implementations, the clinician may simply input the desired impedance change Z_{DROP} by way of the control panel 203 and allow the ESU controller 220 to calculate the impedance set point Z_{SET} in the manner described above and below.

Accordingly, and referring to FIG. 15, the ESU controller 220 may be employed in a tissue ablation procedure that proceeds as follows. The ESU controller 220 may receive the settings for the ablation procedure by way of, for example, the control panel 203 (step S21). Such settings may include, for example, the maximum power level (e.g. 40 W), the duration of the power application (e.g. 20 seconds), the sub-surface temperature set point T_{SET} (e.g.) 65° C.) and the electrode configuration. The ablation electrode (e.g. tip electrode 106) may be advanced to the target tissue (e.g. tissue in the left atrium) before or after the settings are input. Once the settings have been input and the ablation electrode is in contact with the target tissue, the ESU controller 220 will apply low level power to tissue and measure the body temperature tissue impedance Z_{BODYTEMP} (step S22). The impedance set point Z_{SET} is then calculated by the ESU controller 220, based on the sub-surface temperature set point T_{SET}, in the manner described above (step S23) and ablation level power delivery begins (step S24). The ESU controller 220 measures the impedance Z_{PROCEDURE} at the ablation electrode during the delivery of ablation level power (step S25). The ESU controller 220 compares the impedance measured during the ablation procedure Z_{PROCEDURE} to the impedance set point Z_{SET} and regulates the power supplied to the electrode, using PID or other suitable control principles, based on the differences therebetween (step S26). Power will continue to be supplied in this manner until the end of the input power duration (steps S27 and S28).

It should be noted here that there will be an abrupt rise in impedance at when tissue transitions from a non-coagulated state to a coagulated state and when tissue vaporizes and pops. The apparatus and methods described above are not using impedance measurements in this manner. Instead, impedance is being used to estimate sub-surface tissue temperature, based on the relationship between impedance and sub-surface tissue, prior to coagulation and at temperature levels below that which results in popping.

It should also be noted here that the use of impedance in the manner described above to regulate sub-surface tissue temperature is not limited to tip electrodes with a hemispherical end surface. The principles described above are also applicable to, for example tip electrodes with other shapes and electrodes that are located proximal of the tip, such as ring and coil electrodes.

Although the present inventions have been described in terms of the preferred embodiments above, numerous modifications and/or additions to the above-described preferred embodiments would be readily apparent to one skilled in the art. By way of example, but not limitation, the present inventions are applicable to systems that employ multiple electrodes to simultaneously transmit coagulation energy to tissue. The present inventions, including some or all of the aspects thereof, may combined in a single system that, for example, is capable of determining electrode/tissue contact and controlling power based on impedance measurements in the manners described above. It is intended that the scope of the present inventions extend to all such modifications and/or additions and that the scope of the present inventions is limited solely by the claims set forth below.

I claim:

1. A method of evaluating electrode/tissue contact, comprising the steps of:
applying energy to the electrode; and
determining the magnitude of a heat transfer property at the electrode.

2. A method as claimed in claim 1, wherein
the step of applying energy comprises applying energy to the electrode at a relatively low level.

3. A method as claimed in claim 1, wherein
the step of applying energy comprises applying about 1 watt for to the electrode for about 2-5 seconds.

4. A method as claimed in claim 1, wherein
the step of determining the magnitude of a heat transfer property comprises determining the magnitude of the thermal resistance at the electrode.

5. A method as claimed in claim 4, wherein the step of determining the magnitude of a heat transfer property comprises:
measuring the change in temperature at the electrode associated with the application of energy; and
dividing the measured temperature change by the energy applied.

6. A method as claimed in claim 1, further comprising the step of:
comparing the determined heat transfer property to stored heat transfer property values that are indicative of contact with tissue and contact with blood.

7. A method as claimed in claim 1, further comprising the step of:
reporting the determined heat transfer property.

8. A method as claimed in claim 1, wherein
the step of applying energy comprises applying energy to the electrode at a level and time period suitable for tissue coagulation.

9. A power supply for use with an electrophysiology device including an electrode, the power supply comprising:
a power generator; and
means for determining the magnitude of a heat transfer property at the electrode while power from the power generator is being supplied to the electrode.

10. A power supply as claimed in claim 9, further comprising:
means for storing heat transfer property magnitudes indicative of various electrode/tissue contact states; and
means for comparing the determined heat transfer property magnitude to the stored heat transfer property magnitudes.

11. A power supply as claimed in claim 10, wherein
the means for storing comprises means for storing heat transfer property magnitudes indicative of various electrode/tissue contact states for various electrode configurations.

12. A power supply as claimed in claim 10, wherein
the means for storing comprises means for storing heat transfer property magnitudes indicative of various electrode/tissue contact states for various electrode configurations and tissue types.

13. A power supply as claimed in claim 9, further comprising:
means for reporting a result of the comparison.

14. A method, comprising the steps of:
measuring impedance reduction as power is supplied to tissue with an electrode; and
controlling power as a function of the measured impedance reduction.

15. A method as claimed in claim 14, wherein
the measuring and controlling steps are performed prior to an impedance increase associated with tissue coagulation and/or tissue popping.

16. A method as claimed in claim 14, wherein
the step of controlling power comprises controlling power based on an empirically determined relationship between subsurface temperature increase from body temperature and impedance reduction.

17. A method as claimed in claim 14, wherein the step of controlling power comprises:
receiving a subsurface temperature set point;
creating an impedance set point based on the difference between body temperature and the temperature set point and a predetermined relationship between subsurface temperature increase and impedance reduction; and
controlling power as a function of the difference between the impedance set point and the measured impedance.

18. A method as claimed in claim 14, wherein
the step of controlling power comprises controlling power based on the relationship between the measured impedance reduction and an expected impedance reduction.

19. A method as claimed in claim 18, wherein
the step of controlling power comprises increasing power to the electrode in response to the measured impedance reduction being less than the expected impedance reduction.

20. A power supply for use with an electrophysiology device including an electrode, the power supply comprising:
a power generator;
means for measuring impedance reduction as power is supplied to the electrode; and
means for controlling power to the electrode as a function of the measured impedance reduction.

21. A power supply as claimed in claim 20, further comprising:
means for storing a relationship between subsurface temperature increase from body temperature and impedance reduction.

22. A power supply as claimed in claim 21, further comprising:
means for receiving a subsurface temperature set point; and
means for calculating an impedance set point that is less than impedance at body temperature based on the subsurface temperature set point and the stored relationship between subsurface temperature increase from body temperature and impedance reduction.

23. A power supply as claimed in claim 22, wherein
the means for controlling comprises means for controlling power to the electrode as a function of the measured impedance reduction and the impedance set point.
